(19) United States
(12) Patent Application Publication
(10) Pub. No.: US 2026/0347178 A1
(43) Pub. Date: Nov. 5, 2026

(54) METHODS AND SYSTEMS FOR INTEGRATING PETROCHEMICAL AND REFINERY OPERATIONS

(71) Applicant: SABIC Global Technologies B.V., Bergen OP Zoom (NL)

(72) Inventors: Vilas Bhagwat Patil, Jubail (SA); Srikant Vasant Badgandi, Bangalore (IN)

(21) Appl. No.: 19/118,607

(22) PCT Filed: Oct. 4, 2023
(86) PCT No.: PCT/IB2023/059972

§ 371 Date: Apr. 4, 2025

(30) Foreign Application Priority Data
Oct. 4, 2022 (EP) 22199483.3

(51) Int. Cl. C01B 3/38; C01B 3/56; C07C 5/327

(52) U.S. Cl. CPC C01B 3/38; C01B 3/56; C07C 5/327; C01B 2203/042; C01B 2203/062; C01B 2203/1241

(57) ABSTRACT

Systems and methods for integrating petrochemical and refinery operations. One such method includes collecting a hydrogen-rich gas stream generated as a byproduct of a petrochemical operation at a petrochemical plant and purifying the hydrogen-rich gas stream to produce a purified hydrogen gas stream. This method further includes supplying the purified hydrogen gas stream to a hydrotreatment unit or a hydrocracking unit at a refinery and collecting an ethane-rich gas stream generated as a byproduct of a refinery operation at the refinery such that the ethane-rich gas stream may be supplied to a steam cracker at the petrochemical plant to produce olefins, such as ethylene and propylene.

### TECHNICAL FIELD

The present disclosure generally relates to systems and methods for integrating petrochemical and refinery operations. More specifically, the present disclosure relates to systems and methods for collecting a hydrogen-rich gas stream generated as a byproduct or off-gas during petrochemical plant operations and supplying the hydrogen gas stream to a hydrotreatment unit or hydrocracking unit at a refinery, while also collecting an ethane-rich gas stream generated as a byproduct or off-gas during a refinery operation and supplying the ethane gas stream to an ethane steam cracker at the petrochemical plant, thereby maximizing production of olefins, such as ethylene and/or propylene, at a petrochemical plant while reducing refinery energy demand.

### BACKGROUND

Hydrogen (H₂) gas may be generated in situ during several petrochemical plant operations, including chlor-alkali production, ethane steam cracking, methyl tertiary butyl ether (MTBE) production, propane and butane dehydrogenation, and catalytic reforming. In particular, hydrogen may be produced as a co-product together with other light gases in the form of hydrogen rich off-gas streams that typically have a hydrogen content ranging from about 80 mol % to about 90 mol %. Typically, excess hydrogen-rich gas streams generated at petrochemical plants are commonly flared or wasted to the atmosphere. By contrast, refineries may have a hydrogen manufacturing unit (HMU) for the generation of hydrogen from natural gas (NG) feedstock. The hydrogen generated by the HMU may be used by hydrotreating and hydrocracking operations at the refinery. However, HMU operation at refineries is energy and feedstock intensive. Accordingly, there is a need to increase the efficiency or synergy of petrochemical and refining operations by utilizing the waste of off-gas byproducts thereby reducing overall feedstock or energy requirements.

### SUMMARY

To address these and other shortcomings in the art, Applicant has developed systems and methods for integrating or synergizing petrochemical and refinery operations so as to meet one of several operational needs, such as increased efficiency, reduction of waste, and increased economic benefit.

In certain embodiments, the method for integrating petrochemical and refinery operations includes the following steps: collecting a hydrogen-rich gas stream or excess hydrogen off-gas stream generated as a byproduct of a petrochemical operation at a petrochemical plant; purifying the hydrogen-rich gas stream or excess hydrogen off-gas stream to produce a purified hydrogen gas stream; supplying the purified hydrogen gas stream to a hydrotreatment unit or a hydrocracking unit at a refinery; collecting an ethane-rich gas stream generated as a byproduct of a refinery operation at the refinery; and supplying the ethane-rich gas stream to an ethane steam cracker at the petrochemical plant to produce ethylene. In at least some embodiments, the hydrogen-rich gas stream may be an off-gas by-product generated by the petrochemical operation and the ethane-rich gas stream may be an off-gas generated by the refinery operation. In some embodiments of the method, substantially all of the hydrogen gas generated as a byproduct of a petrochemical operation at petrochemical plant, that would typically be wasted or flared, is collected and supplied to a hydrotreatment unit or a hydrocracking unit at refinery in order to reduce the natural gas or other energy demands of one or more refinery operations.

In certain embodiments, the petrochemical operation may be chlor-alkali production, ethane steam cracking, methyl tertiary butyl ether (MTBE) production, propane and butane dehydrogenation, catalytic reforming, or any combination thereof. In some embodiments, the refinery operation may include a cracking operation, such as fluid catalytic cracking.

In some embodiments, the hydrogen-rich gas stream may be purified by processing the hydrogen-rich gas stream through a pressure swing adsorption unit or through a guard bed. In some embodiments, the purified hydrogen gas stream comprises 99.9 weight percent of hydrogen. The purified hydrogen gas stream may contain greater than 90 wt. % hydrogen gas, or greater than 95 wt. % hydrogen gas, or greater than 98 wt. % hydrogen gas, or at least 99 wt. % hydrogen gas or at least 99.9 wt. % hydrogen gas.

In some embodiments, prior to the step of supplying the purified hydrogen gas stream to the hydrotreatment unit or a hydrocracking unit, the method may include mixing the purified hydrogen gas stream with a hydrogen gas stream produced by the hydrogen manufacturing unit at the refinery to generate a combined hydrogen gas stream. In such embodiments, the combined hydrogen gas stream may be fed to the hydrotreatment unit or the hydrocracking unit. In at least some instances, the hydrotreatment unit or the hydrocracking unit may be coupled with the hydrogen manufacturing unit.

In at least some embodiments, the method may include reducing the demand load on a hydrogen manufacturing unit at the refinery in response to supplying the purified hydrogen gas stream to the hydrotreatment unit or the hydrocracking unit at a refinery. In some embodiments, the method may further include reducing natural gas consumption at the hydrogen manufacturing unit coupled with the hydrotreatment unit or the hydrocracking unit at the refinery in response to supplying the purified hydrogen gas stream to the hydrotreatment unit or the hydrocracking unit. In at least some embodiments, the method may further include reducing carbon dioxide emission at the hydrogen manufacturing unit coupled with the hydrotreatment unit or the hydrocracking unit at the refinery in response to supplying the purified hydrogen gas stream to the hydrotreatment unit or the hydrocracking unit. Embodiments may include supplying the ethane-rich gas stream to an ethane steam cracker located at the petrochemical plant such that propylene is generated in addition to ethylene.

The present disclosure also provides an integrated system for petrochemical and refinery operations. The system may include a petrochemical plant and a refinery unit. The petrochemical plant may include a hydrogen off-gas collection unit operable to collect a hydrogen-rich gas stream generated as a byproduct of a petrochemical operation. The petrochemical plant may further include a hydrogen off-gas purification unit operable to purify the hydrogen-rich gas stream to produce a purified hydrogen gas stream and an ethane steam cracker operable to receive an ethane-rich gas stream. The refinery unit may include a hydrotreatment unit or a hydrocracking unit operable to receive the purified hydrogen gas stream from the hydrogen off-gas purification unit and an ethane off-gas collection unit operable to collect the ethane-rich gas stream generated as a byproduct of a refinery operation and in fluid communication with the ethane steam cracker.

In at least some embodiments, the hydrogen-rich gas stream may be an off-gas by-product generated by the petrochemical operation and the ethane-rich gas stream may be an off-gas generated by the refinery operation. In some embodiments, the refinery operation may include a cracking operation, such as fluid catalytic cracking.

In some embodiments, the petrochemical plant may further include at least one processing unit selected from the group consisting of a chlor-alkali production unit, an ethane steam cracking unit, a methyl tertiary butyl ether (MTBE) production unit, a propane and butane dehydrogenation unit, a catalytic reforming unit, and any combination thereof. Accordingly, in at least some embodiments, the petrochemical operation in the system may be a chlor-alkali production, an ethane steam cracking, a methyl tertiary butyl ether (MTBE) production, a propane and butane dehydrogenation, a catalytic reforming, or any combination thereof. The at least one processing unit may be coupled to the hydrogen off-gas collection unit.

In some embodiments, the hydrogen off-gas purification unit may include a pressure swing adsorption unit or one or more guard beds. The purified hydrogen gas stream may contain greater than 90 wt. % hydrogen gas, or greater than 95 wt. % hydrogen gas, or greater than 98wt. % hydrogen gas, or at least 99 wt. % hydrogen gas or at least 99.9 wt. % hydrogen gas.

In some embodiments, the refinery may further include a hydrogen manufacturing unit operable to produce hydrogen gas from natural gas. The hydrogen manufacturing unit (HMU) may be coupled to the hydrotreatment unit or the hydrocracking unit. The hydrotreatment unit or the hydrocracking unit may be operable to receive the purified hydrogen gas stream such that the demand load on the hydrogen manufacturing unit is reduced. In some embodiments, the hydrotreatment unit or the hydrocracking unit may be operable to receive a hydrogen fuel mixture comprising the purified hydrogen gas stream, or a portion thereof, and a hydrogen gas stream produced by the hydrogen manufacturing unit.

Embodiments of the system include a hydrotreatment unit or a hydrocracking unit operable to receive the purified hydrogen gas stream so as to reduce natural gas consumption or carbon dioxide emissions at the hydrogen manufacturing unit.

In at least some embodiments, the ethane steam cracker is operable to produce ethylene and propylene from the ethane-rich gas stream and the ethane off-gas collection unit is operable to send ethane-rich gas stream to the ethane steam cracker at the petrochemical plant.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a graphical representation of a system for integrating petrochemical and refinery operations, according to an exemplary embodiment of the present disclosure.

### DETAILED DESCRIPTION

The present disclosure describes various embodiments related to processes, methods, and systems for integrating petrochemical and refinery operations. Further embodiments may be described and disclosed.

In the following description, numerous details are set forth in order to provide a thorough understanding of the various embodiments. In other instances, well-known processes, devices, and systems may not have been described in particular detail in order not to unnecessarily obscure the various embodiments. Additionally, illustrations of the various embodiments may omit certain features or details in order to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” refers to a range of values including the specified value, which a person of ordinary skill in the art would consider reasonably similar to the specified value. In embodiments, “about” refers to values within a standard deviation using measurements generally acceptable in the art. In one non-limiting embodiment, when the term “about” is used with a particular value, then “about” refers to a range extending to ±10% of the specified value, alternatively ±5% of the specified value, or alternatively ±1% of the specified value, or alternatively ±0.5% of the specified value. In embodiments, “about” refers to the specified value.

The terms “reducing,” “reduced,” or any variation thereof, when used in the claims and/or the specification includes any measurable decrease or complete removal to achieve a desired result.

The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, that includes the component. In a non-limiting example, 10 grams of a component in 100 grams of the material is 10 wt. % of such component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Disclosed here are systems and methods for integrating petrochemical and refinery operations. In particular, the presently disclosed systems and methods provide for the synergistic use of off-gases as feed streams in petrochemical and refinery operations thereby maximizing light olefin production at a petrochemical plant while simultaneously reducing refinery energy demand. More specifically, the present disclosure involves systems and methods for collecting excess hydrogen-rich gas stream generated as a byproduct or off-gas during petrochemical plant operations and supplying the hydrogen gas stream to a hydrotreatment unit or hydrocracking unit at a refinery, while also collecting an ethane-rich gas stream generated as a byproduct or off-gas during a refinery operation and supplying the ethane gas stream to an ethane steam cracker at the petrochemical plant.

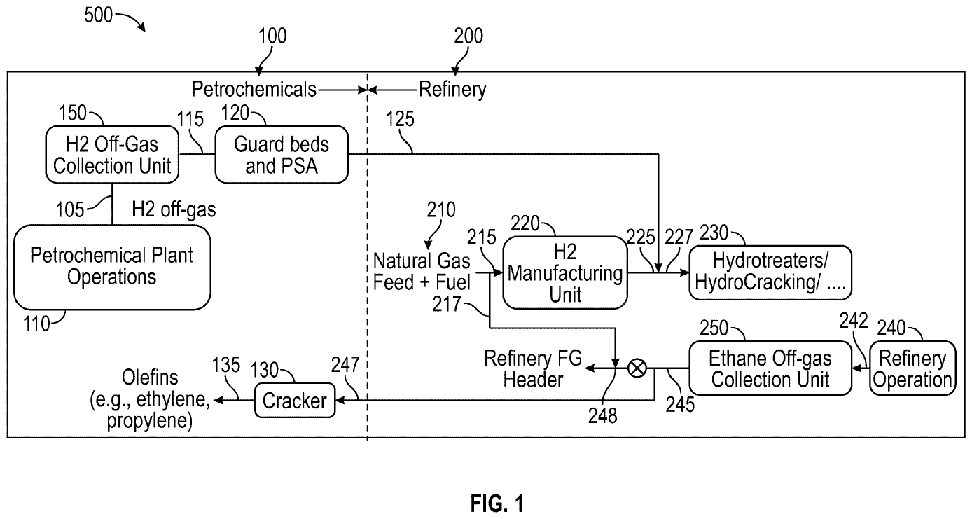

FIG. 1 is a schematic depicting a system 500 for integrating petrochemical and refinery operations, according to an exemplary embodiment of the present disclosure. As depicted in FIG. 1, system 500 may include a petrochemical plant 100 and a refinery unit 200. The petrochemical plant 100 may include one or more petrochemical operations 110 capable of generating hydrogen off-gas 105. Non-limiting examples of petrochemical operations 110 may include a chlor-alkali production operation, an ethane steam cracking operation, a methyl tertiary butyl ether (MTBE) production operation, a propane and butane dehydrogenation operation, and a catalytic reforming operation. In at least some embodiments, the one or more petrochemical operations 110 at the petrochemical plant 100 may be in the form of a processing unit 110 selected from the group consisting of a chlor-alkali production unit, an ethane steam cracking unit, a methyl tertiary butyl ether (MTBE) production unit, a propane and butane dehydrogenation unit, a catalytic reforming unit, and any combination thereof.

In at least some embodiments, the processing unit 110 may be an electrified cracker which generates a substantial excess of hydrogen off-gas. For example, the electrified cracker may be employed as part of a de-carbonization strategy at the petrochemical plant. In such instances, the substantial excess hydrogen off-gas produced by an electrified cracker may not be useable as a fuel within the cracker complex. The present disclosure provides a system 500 having improved utilization of hydrogen off-gas that increases the efficiency of system 500.

The petrochemical plant 100 may include a hydrogen off-gas collection unit 150 coupled to the one or more petrochemical operations 110 or processing unit 110. The hydrogen off-gas collection unit 150 is operable to collect the hydrogen-rich off-gas stream 105 generated as a byproduct of the petrochemical operation 110 or operation of processing unit 110. The hydrogen-rich off-gas stream 105 may be contaminated with salts, chlorides, alkanes, alkenes, and other contaminants depending on the process technology deployed and the feed characteristics. Instead of flaring or wasting the hydrogen-rich off-gas stream 105, the presently disclosed system 500 includes a means for purifying the hydrogen-rich off-gas stream 105 so that it is suitable for utilization by hydrotreating or hydrocracking operations at a refinery.

Accordingly, the petrochemical plant 100 may further include a hydrogen off-gas purification unit 120 coupled to the hydrogen off-gas collection unit 150. The hydrogen off-gas purification unit 120 is operable to purify the hydrogen-rich off-gas stream 105 collected by the hydrogen off-gas collection unit 150 to produce a purified hydrogen gas stream 125. In some embodiments, the hydrogen off-gas purification unit 120 may include a pressure swing adsorption unit and/or one or more guard beds. The purified hydrogen gas stream 125 may contain greater than 90 wt. % hydrogen gas, or greater than 95 wt. % hydrogen gas, or greater than 98 wt. % hydrogen gas, or at least 99 wt. % hydrogen gas or at least 99.9 wt. % hydrogen gas.

As depicted in FIG. 1, petrochemical plant 100 may further include an ethane steam cracker 130 operable to receive an ethane-rich gas stream and produce light olefins 135, such as ethylene and propylene.

The refinery unit 200 may include a hydrotreatment or hydrocracking unit 230 operable to receive a hydrogen gas stream. For example, hydrocracking unit 230 may be configured to upgrade a vacuum gasoil feedstock through cracking while injecting hydrogen gas to produce diesel or kerosene products. In order to provide the hydrogen gas stream 227 to the hydrotreater or hydrocracking unit 230, refinery unit 200 may include a hydrogen manufacturing unit (HMU) 220 operable to produce a hydrogen gas stream 225 from natural gas, naphtha, and/or liquefied petroleum gas (LPG) feedstock 210. In particular, HMU 220 is operable to receive a natural gas or fuel feed stream 215 and produce a hydrogen gas stream 225. The hydrogen manufacturing unit (HMU) 220 may be coupled to the hydrotreatment unit or the hydrocracking unit 230 for the purpose of feeding a hydrogen gas stream 227 to the hydrotreatment or hydrocracking unit 230.

The hydrotreatment unit or the hydrocracking unit 230 is operable to receive the purified hydrogen gas stream 125 produced by the hydrogen off-gas purification unit 120 at the petrochemical plant 100 such that the demand load on the hydrogen manufacturing unit 220 is reduced. Therefore, system 500 includes a means for transporting purified hydrogen gas stream 125 from petrochemical plant 100 to hydrotreatment or hydrocracking unit 230 at refinery 200. The means for transporting the purified hydrogen gas stream 125 from petrochemical plant 100 to hydrotreatment or hydrocracking unit 230 at the refinery 200 may include any known in the art, including a pipeline or vehicular transport, such as by truck or railcar, and may include any necessary intermediate facilities or storage tanks. In some embodiments, the hydrotreatment or hydrocracking unit 230 may be operable to receive a hydrogen fuel mixture 227 comprising the purified hydrogen gas stream 125 generated at the petrochemical plant 100, or a portion thereof, and a hydrogen gas stream 225 produced by the hydrogen manufacturing unit 220. In at least some embodiments, system 500 includes a hydrotreatment or hydrocracking unit 230 operable to receive the purified hydrogen gas stream 125 from the petrochemical plant 100 so as to reduce natural gas consumption or carbon dioxide emissions at the hydrogen manufacturing unit 220.

The refinery unit 200 may also include one or more refinery operations 240 capable of producing an ethane-rich off-gas stream 242. In some embodiments, the refinery operation 240 may include a cracking operation, such as fluid catalytic cracking. Refinery unit 200 may further include an ethane off-gas collection unit 250 operable to collect the ethane-rich gas stream 242 generated as a byproduct of a refinery operation 240. A portion 248 of the ethane-rich off-gas 245 collected by ethane off-gas collection unit may be utilized a fuel in the refinery fuel gas header. However, as depicted in FIG. 1, a portion 247 of the ethane-rich off-gas stream 245 from ethane off-gas collection unit 250 is diverted as a feedstock to ethane cracker 130 at the petrochemical plant 100.

The ethane steam cracker 130 at petrochemical plant 100 is operable to receive the ethane-rich off-gas stream 247, or a portion thereof, from the ethane off-gas collection unit 250 and/or refinery operation 240 at the refinery unit 200. The ethane stream cracker 130 is further operable to produce light olefins, such as ethylene and propylene, from the ethane-rich gas stream 247 received from the refinery unit 200. Therefore, system 500 includes a means for transporting the ethane-rich gas stream 245 from the ethane off-gas collection unit 250 or refinery operation 240 at refinery 200 to the ethane steam cracker 130 at petrochemical plant 100. The means for transporting the ethane-rich gas stream gas stream 245 from refinery 200 to ethane steam cracker 130 at the petrochemical plant 100 may include any known in the art, including a pipeline or vehicular transport, such as by truck or railcar, and may include any necessary intermediate facilities or storage tanks. In at least some embodiments, the ethane off-gas collection unit 250 may be in fluid communication with the ethane steam cracker 130. In some embodiments, the ethane off-gas collection unit 250 is operable to send ethane-rich gas stream 245 to the ethane steam cracker 130 at the petrochemical plant 100. The presently disclosed system 500 is operable to increase light olefin production at the petrochemical plant 100 by providing an additional ethane-rich gas stream to the ethane stream cracker 130 derived from off-gas waste generated by one or more refinery operations 240.

In at least some embodiments, a portion of the ethane off-gas 248 from ethane off-gas collection unit 250 or refinery operation 240 may be used to supply the refinery fuel gas header. A portion 217 of the natural gas and fuel feed 215 to the hydrogen manufacturing unit 220 may also be diverted to supply the fuel gas header at the refinery 200.

In some embodiments, the hydrogen off-gases generated at the petrochemical plant 100, or a purified hydrogen gas stream generated therefrom, may be swapped for an energetically or economically equivalent amount of ethane-rich off-gas generated by one or more refinery operations 240 at the refinery 200. In this manner, a synergism between petrochemical plant 100 operations and refinery 200 operations may be established.

A method for integrating petrochemical and refinery operations is illustrated by the system described in FIG. 1. For example, the method may include collecting the hydrogen-rich gas stream 105 generated as a byproduct of a petrochemical operation 110 at a petrochemical plant 100 and purifying the hydrogen-rich gas stream to produce a purified hydrogen gas stream 125. The method may further include supplying the purified hydrogen gas stream 125 to a hydrotreatment unit or a hydrocracking unit 230 at a refinery 200 and collecting an ethane-rich gas stream 242 generated as a byproduct of a refinery operation 240 at the refinery 200. The method may further include supplying the ethane-rich gas stream 247, or a portion thereof, to an ethane steam cracker 130 at the petrochemical plant 100 to produce light olefins such as ethylene and propylene.

The present disclosure provides systems and methods for the integration of petrochemical and refinery operations such that ethylene and/or propylene production is increased at a petrochemical plant while reducing energy demand at a refinery. In particular, the present disclosure provides a method for integrating petrochemical and refinery operations, the method comprising: collecting excess hydrogen-rich off-gas streams generated as a byproduct of a petrochemical operation at a petrochemical plant; purifying the hydrogen-rich gas stream to produce a purified hydrogen gas stream; supplying the purified hydrogen gas stream to a hydrotreatment unit or a hydrocracking unit at a refinery; collecting an ethane-rich gas stream generated as a byproduct of a refinery operation at the refinery; and supplying the ethane-rich gas stream to an ethane steam cracker at the petrochemical plant to produce ethylene. The petrochemical operation in the preceding method embodiment may be selected from the group consisting of chlor-alkali production, ethane steam cracking, methyl tertiary butyl ether (MTBE) production, propane and butane dehydrogenation, catalytic reforming, and any combination thereof.

In the preceding method embodiment, the hydrogen-rich gas stream may be purified by processing the hydrogen-rich gas stream through a pressure swing adsorption unit. In the preceding method embodiment, the hydrogen-rich gas stream may be purified by processing the hydrogen-rich gas stream through a guard bed. In the preceding method embodiment, the purified hydrogen gas stream may comprise at least 99.9 weight percent of hydrogen.

The preceding method embodiment may further include in response to supplying the purified hydrogen gas stream to the hydrotreatment unit or a hydrocracking unit, reducing demand load on a hydrogen manufacturing unit at the refinery. The preceding method embodiment may further include, prior to the step of supplying the purified hydrogen gas stream to the hydrotreatment unit or a hydrocracking unit, mixing the purified hydrogen gas stream with a hydrogen gas stream produced by the hydrogen manufacturing unit at the refinery to generate a combined hydrogen gas stream, the combined hydrogen gas stream fed to the hydrotreatment unit or the hydrocracking unit. In the preceding method embodiment, the hydrotreatment unit or the hydrocracking unit may be coupled with the hydrogen manufacturing unit.

The preceding method embodiment may further include, in response to supplying the purified hydrogen gas stream to the hydrotreatment unit or the hydrocracking unit, reducing natural gas consumption at the hydrogen manufacturing unit coupled with the hydrotreatment unit or the hydrocracking unit at the refinery. The preceding method embodiment may further include in response to supplying the purified hydrogen gas stream to the hydrotreatment unit or the hydrocracking unit, reducing carbon dioxide emission at the hydrogen manufacturing unit coupled with the hydrotreatment unit or the hydrocracking unit at the refinery.

In the preceding method embodiment, the hydrogen-rich gas stream may be an off-gas by-product generated by the petrochemical operation. In the preceding method embodiment, the refinery operation may include a cracking operation. In the preceding method embodiment, the cracking operation may be fluid catalytic cracking. In the preceding method embodiment, the ethane-rich gas stream may be an off-gas generated by the refinery operation. In the preceding method embodiment, supplying the ethane-rich gas stream to an ethane steam cracker located at the petrochemical plant may produce propylene in addition to the ethylene.

The present disclosure also provides an integrated system for petrochemical and refinery operations operable to increase ethylene and/or propylene production at a petrochemical plant while reducing energy demand at a refinery. The integrated system for petrochemical and refinery operations may comprise: a petrochemical plant comprising: a hydrogen off-gas collection unit operable to collect a hydrogen-rich gas stream generated as a byproduct of a petrochemical operation; a hydrogen off-gas purification unit operable to purify the hydrogen-rich gas stream to produce a purified hydrogen gas stream; and an ethane steam cracker operable to receive an ethane-rich gas stream; and a refinery unit comprising: a hydrotreatment unit or a hydrocracking unit operable to receive the purified hydrogen gas stream from the hydrogen off-gas purification unit; and an ethane off-gas collection unit operable to collect the ethane-rich gas stream generated as a byproduct of a refinery operation and in fluid communication with the ethane steam cracker.

In the preceding system embodiment, the petrochemical operation may be chlor-alkali production, an ethane steam cracking, a methyl tertiary butyl ether (MTBE) production, a propane and butane dehydrogenation, a catalytic reforming, or any combination thereof. The petrochemical plant in the preceding system embodiment may further include at least one processing unit selected from the group consisting of a chlor-alkali production unit, an ethane steam cracking unit, a methyl tertiary butyl ether (MTBE) production unit, a propane and butane dehydrogenation unit, a catalytic reforming unit, or any combination thereof, the at least one processing unit coupled to the hydrogen off-gas collection unit.

In the preceding system embodiment, the hydrogen off-gas purification unit may comprise a pressure swing adsorption unit. In the preceding system embodiment, the hydrogen off-gas purification unit may comprise one or more guard beds. In the preceding system embodiment, the purified hydrogen gas stream may comprise at least 99.9 wt. % hydrogen gas. The refinery in the preceding system embodiment may further comprise a hydrogen manufacturing unit operable to produce hydrogen gas from natural gas and coupled to the hydrotreatment unit or the hydrocracking unit. In the preceding system embodiment, the hydrotreatment unit or the hydrocracking unit may be operable to receive the purified hydrogen gas stream such that the demand load on the hydrogen manufacturing unit is reduced. In the preceding system embodiment, the hydrotreatment unit or the hydrocracking unit may be operable to receive a hydrogen fuel mixture, the hydrogen fuel mixture comprising the purified hydrogen gas stream, or a portion thereof, and a hydrogen gas stream produced by the hydrogen manufacturing unit.

In the preceding system embodiment, the hydrotreatment unit or the hydrocracking unit may be operable to receive the purified hydrogen gas stream and reduce natural gas consumption at the hydrogen manufacturing unit. In the preceding system embodiment, the hydrotreatment unit or the hydrocracking unit may be operable to receive the purified hydrogen gas stream and reduce carbon dioxide emissions at the hydrogen manufacturing unit. In the preceding system embodiment, the hydrogen-rich gas stream may be an off-gas generated by the petrochemical operation.

In the preceding system embodiment, the ethane steam cracker may be operable to produce ethylene and propylene from the ethane-rich gas stream. In the preceding system embodiment, the refinery operation may be a cracking operation. In the preceding system embodiment, the cracking operation may be fluid catalytic cracking. In the preceding system embodiment, the ethane off-gas collection unit may be operable to send the ethane-rich gas stream to the ethane steam cracker at the petrochemical plant.

The present disclosure also provides a method for the integration of petrochemical and refinery operations resulting in increased ethylene and/or propylene production at a petrochemical plant while reducing energy demand at a refinery. In particular, the present disclosure provides a method for integrating petrochemical and refinery operations, the method comprising: collecting a hydrogen-rich gas stream generated as a byproduct of a petrochemical operation at a petrochemical plant; purifying the hydrogen-rich gas stream to produce a purified hydrogen gas stream; supplying the purified hydrogen gas stream to a hydrotreatment unit or a hydrocracking unit at a refinery; collecting an ethane-rich gas stream generated as a byproduct of a refinery operation at the refinery; and supplying the ethane-rich gas stream to an ethane steam cracker at the petrochemical plant to produce ethylene. The petrochemical operation in the preceding method embodiment may be selected from the group consisting of chlor-alkali production, ethane steam cracking, methyl tertiary butyl ether (MTBE) production, propane and butane dehydrogenation, catalytic reforming, and any combination thereof.

In any of the preceding method embodiments, the hydrogen-rich gas stream may be purified by processing the hydrogen-rich gas stream through a pressure swing adsorption unit. In any of the preceding method embodiments, the hydrogen-rich gas stream may be purified by processing the hydrogen-rich gas stream through a guard bed. In any of the preceding method embodiments, the purified hydrogen gas stream may comprise at least 99.9 weight percent of hydrogen.

Any of the preceding method embodiments may further include, in response to supplying the purified hydrogen gas stream to the hydrotreatment unit or a hydrocracking unit, reducing demand load on a hydrogen manufacturing unit at the refinery. Any of the preceding method embodiments may further include, prior to the step of supplying the purified hydrogen gas stream to the hydrotreatment unit or a hydrocracking unit, mixing the purified hydrogen gas stream with a hydrogen gas stream produced by the hydrogen manufacturing unit at the refinery to generate a combined hydrogen gas stream, the combined hydrogen gas stream fed to the hydrotreatment unit or the hydrocracking unit. In any of the preceding method embodiments, the hydrotreatment unit or the hydrocracking unit may be coupled with the hydrogen manufacturing unit.

Any of the preceding method embodiments may further include, in response to supplying the purified hydrogen gas stream to the hydrotreatment unit or the hydrocracking unit, reducing natural gas consumption at the hydrogen manufacturing unit coupled with the hydrotreatment unit or the hydrocracking unit at the refinery. Any of the preceding method embodiments may further include in response to supplying the purified hydrogen gas stream to the hydrotreatment unit or the hydrocracking unit, reducing carbon dioxide emission at the hydrogen manufacturing unit coupled with the hydrotreatment unit or the hydrocracking unit at the refinery.

In any of the preceding method embodiments, the hydrogen-rich gas stream may be an off-gas by-product generated by the petrochemical operation. In any of the preceding method embodiments, the refinery operation may include a cracking operation. In any of the preceding method embodiments, the cracking operation may be fluid catalytic cracking. In any of the preceding method embodiments, the ethane-rich gas stream may be an off-gas generated by the refinery operation. In any of the preceding method embodiments, supplying the ethane-rich gas stream to an ethane steam cracker located at the petrochemical plant may produce propylene in addition to the ethylene.

The present disclosure also provides an integrated system for petrochemical and refinery operations operable to increase ethylene and/or propylene production at a petrochemical plant while reducing energy demand at a refinery. The integrated system for petrochemical and refinery operations may comprise: a petrochemical plant comprising: a hydrogen off-gas collection unit operable to collect a hydrogen-rich gas stream generated as a byproduct of a petrochemical operation; a hydrogen off-gas purification unit operable to purify the hydrogen-rich gas stream to produce a purified hydrogen gas stream; and an ethane steam cracker operable to receive an ethane-rich gas stream; and a refinery unit comprising: a hydrotreatment unit or a hydrocracking unit operable to receive the purified hydrogen gas stream from the hydrogen off-gas purification unit; and an ethane off-gas collection unit operable to collect the ethane-rich gas stream generated as a byproduct of a refinery operation and in fluid communication with the ethane steam cracker.

In the preceding system embodiment, the petrochemical operation may be chlor-alkali production, an ethane steam cracking, a methyl tertiary butyl ether (MTBE) production, a propane and butane dehydrogenation, a catalytic reforming, or any combination thereof. The petrochemical plant in any of the preceding system embodiments may further include at least one processing unit selected from the group consisting of a chlor-alkali production unit, an ethane steam cracking unit, a methyl tertiary butyl ether (MTBE) production unit, a propane and butane dehydrogenation unit, a catalytic reforming unit, or any combination thereof, the at least one processing unit coupled to the hydrogen off-gas collection unit.

In any of the preceding system embodiments, the hydrogen off-gas purification unit may comprise a pressure swing adsorption unit. In any of the preceding system embodiments, the hydrogen off-gas purification unit may comprise one or more guard beds. In any of the preceding system embodiments, the purified hydrogen gas stream may comprise at least 99.9 wt. % hydrogen gas. The refinery in any of the preceding system embodiments may further comprise a hydrogen manufacturing unit operable to produce hydrogen gas from natural gas and coupled to the hydrotreatment unit or the hydrocracking unit. In any of the preceding system embodiments, the hydrotreatment unit or the hydrocracking unit may be operable to receive the purified hydrogen gas stream such that the demand load on the hydrogen manufacturing unit is reduced. In any of the preceding system embodiments, the hydrotreatment unit or the hydrocracking unit may be operable to receive a hydrogen fuel mixture, the hydrogen fuel mixture comprising the purified hydrogen gas stream, or a portion thereof, and a hydrogen gas stream produced by the hydrogen manufacturing unit.

In any of the preceding system embodiments, the hydrotreatment unit or the hydrocracking unit may be operable to receive the purified hydrogen gas stream and reduce natural gas consumption at the hydrogen manufacturing unit. In any of the preceding system embodiments, the hydrotreatment unit or the hydrocracking unit may be operable to receive the purified hydrogen gas stream and reduce carbon dioxide emissions at the hydrogen manufacturing unit. In any of the preceding system embodiments, the hydrogen-rich gas stream may be an off-gas generated by the petrochemical operation.

In any of the preceding system embodiments, the ethane steam cracker may be operable to produce ethylene and propylene from the ethane-rich gas stream. In any of the preceding system embodiments, the refinery operation may be a cracking operation. In any of the preceding system embodiments, the cracking operation may be fluid catalytic cracking. In any of the preceding system embodiments, the ethane off-gas collection unit may be operable to send the ethane-rich gas stream to the ethane steam cracker at the petrochemical plant.

### EXAMPLES

The example provided below illustrates selected aspects of the various methods and systems of integrating petrochemical plant and refinery operations.

### Example 1

Implementation of the exemplary embodiment of the system provided in FIG. 1 results in a quantifiable savings of natural gas demand on the hydrogen manufacturing unit (HMU) as well as a corresponding reduction in CO₂ emissions due to the decreased load on the HMU in addition to providing additional feedstock to the ethane cracker at the petrochemical plant. In particular, implementation of the presently disclosed system produces 70 tons/day (t/d) hydrogen off-gas 105 from petrochemical plant operation 110 resulting in 56 t/d purified hydrogen gas stream 125 available to be fed to the hydrotreatment or hydrocracking unit 230 at the refinery 200. The resultant reduced demand on the HMU 220 produced a natural gas savings of 90 t/d even after fulfilling the fuel gas header make-up in the refinery by natural gas. At the same time, implementation of the presently disclosed system and method yielded 160 t/d of ethane-rich refinery off-gas produced by refinery operation 140 that was available to be fed to the ethane cracker 130 at petrochemical plant 100. This results in a direct savings of about 90 t/d of natural gas that would otherwise be fed to the hydrogen manufacturing unit, resulting in about 200 t/d of carbon dioxide emission reductions.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A method for integrating petrochemical and refinery operations, the method comprising:
collecting a hydrogen-rich gas stream generated as a byproduct of a petrochemical operation at a petrochemical plant;
purifying the hydrogen-rich gas stream to produce a purified hydrogen gas stream;
supplying the purified hydrogen gas stream to a hydrotreatment unit or a hydrocracking unit at a refinery;
collecting an ethane-rich gas stream generated as a byproduct of a refinery operation at the refinery; and
supplying the ethane-rich gas stream to an ethane steam cracker at the petrochemical plant to produce ethylene.

2. The method according to claim 1, wherein the petrochemical operation is selected from the group consisting of chlor-alkali production, ethane steam cracking, methyl tertiary butyl ether (MTBE) production, propane and butane dehydrogenation, catalytic reforming, and any combination thereof.

3. The method according to claim 1, wherein the hydrogen-rich gas stream is purified by processing the hydrogen-rich gas stream through a pressure swing adsorption unit or a guard bed, the purified hydrogen gas stream comprising 99.9 weight percent of hydrogen.

4. The method according to claim 1, further comprising:
in response to supplying the purified hydrogen gas stream to the hydrotreatment unit or a hydrocracking unit, reducing demand load on a hydrogen manufacturing unit at the refinery.

5. The method according to claim 1, further comprising:
prior to the step of supplying the purified hydrogen gas stream to the hydrotreatment unit or a hydrocracking unit, mixing the purified hydrogen gas stream with a hydrogen gas stream produced by the hydrogen manufacturing unit at the refinery to generate a combined hydrogen gas stream, the combined hydrogen gas stream fed to the hydrotreatment unit or the hydrocracking unit; wherein the hydrotreatment unit or the hydrocracking unit is coupled with the hydrogen manufacturing unit.

6. The method according to claim 1, further comprising:
in response to supplying the purified hydrogen gas stream to the hydrotreatment unit or the hydrocracking unit, reducing natural gas consumption at the hydrogen manufacturing unit coupled with the hydrotreatment unit or the hydrocracking unit at the refinery.

7. The method according to claim 1, further comprising:
in response to supplying the purified hydrogen gas stream to the hydrotreatment unit or the hydrocracking unit, reducing carbon dioxide emission at the hydrogen manufacturing unit coupled with the hydrotreatment unit or the hydrocracking unit at the refinery.

8. The method according to claim 1, wherein the hydrogen-rich gas stream is an off-gas by-product generated by the petrochemical operation, wherein the ethane-rich gas stream is an off-gas generated by the refinery operation, and wherein supplying the ethane-rich gas stream to an ethane steam cracker located at the petrochemical plant produces propylene in addition to the ethylene.

9. An integrated system for petrochemical and refinery operations, the system comprising:
a petrochemical plant comprising:
a hydrogen off-gas collection unit operable to collect a hydrogen-rich gas stream generated as a byproduct of a petrochemical operation;
a hydrogen off-gas purification unit operable to purify the hydrogen-rich gas stream to produce a purified hydrogen gas stream; and
a steam cracker operable to receive an ethane-rich gas stream; and
a refinery unit comprising:
a hydrotreatment unit or a hydrocracking unit operable to receive the purified hydrogen gas stream from the hydrogen off-gas purification unit; and
an ethane off-gas collection unit operable to collect the ethane-rich gas stream generated as a byproduct of a refinery operation and in fluid communication with the steam cracker.

10. The system according to claim 9, wherein the petrochemical operation is selected from the group consisting of a chlor-alkali production, an ethane steam cracking, a methyl tertiary butyl ether (MTBE) production, a propane and butane dehydrogenation, a catalytic reforming, and any combination thereof.

11. The system according to claim 10, wherein the petrochemical plant further comprises:
at least one processing unit selected from the group consisting of a chlor-alkali production unit, an ethane steam cracking unit, a methyl tertiary butyl ether (MTBE) production unit, a propane and butane dehydrogenation unit, a catalytic reforming unit, or any combination thereof, the at least one processing unit coupled to the hydrogen off-gas collection unit.

12. The system according to claim 9, wherein the hydrogen off-gas purification unit comprises a pressure swing adsorption unit or one or more guard beds, and wherein the purified hydrogen gas stream comprises 99.9 wt. % hydrogen gas.

13. The system according to claim 9, wherein the refinery further comprises:
a hydrogen manufacturing unit operable to produce hydrogen gas from natural gas and coupled to the hydrotreatment unit or the hydrocracking unit, wherein the hydrotreatment unit or the hydrocracking unit is operable to receive the purified hydrogen gas stream such that the demand load on the hydrogen manufacturing unit is reduced.

14. The system according to claim 9, wherein the hydrotreatment unit or the hydrocracking unit is operable to receive a hydrogen fuel mixture, the hydrogen fuel mixture comprising the purified hydrogen gas stream, or a portion thereof, and a hydrogen gas stream produced by the hydrogen manufacturing unit.

15. The system according to claim 9, wherein the hydrotreatment unit or the hydrocracking unit is operable to receive the purified hydrogen gas stream and reduce natural gas consumption or carbon dioxide emissions at the hydrogen manufacturing unit, wherein the steam cracker is operable to produce ethylene and propylene from the ethane-rich stream, and wherein the ethane off-gas collection unit is operable to send ethane-rich stream to the steam cracker at the petrochemical plant.
